(19) United States
(12) Patent Application Publication
(10) Pub. No.: US 2024/0206486 A1
(43) Pub. Date: Jun. 27, 2024

(54) SYSTEMS AND METHODS FOR STORING PRODUCE

(71) Applicant: Maersk Container Industry A/S, Tinglev (DK)

(72) Inventors: Niels Nielsen POULSEN, Tinglev (DK); Henriette Wase HANSEN, Tinglev (DK)

(21) Appl. No.: 18/598,345

(22) Filed: Mar. 7, 2024

Related U.S. Application Data
(63) Continuation of application No. PCT/EP2022/074610, filed on Sep. 5, 2022.

(30) Foreign Application Priority Data
Sep. 8, 2021 (DK) PA202100871

(51) Int. Cl. A23B 7/152; G01N 33/02

(52) U.S. Cl. CPC A23B 7/152; G01N 33/025

(57) ABSTRACT

Disclosed is a method for maintaining produce in an unripened state in a storage unit. The storage unit includes a space in which the produce is stored. The method includes monitoring a respiration rate of the produce and, in response to a change in the monitored respiration rate, causing an action to be performed to facilitate maintenance of the unripened state.

### CROSS-REFERENCE TO RELATED APPLICATIONS

This application is a continuation under 35 U.S.C. § 120 of International Application No. PCT/EP2022/074610, filed Sep. 5, 2022, which claims priority to DK Application No. PA202100871, filed Sep. 8, 2021, under 35 U.S.C. § 119(a). Each of the above-referenced patent applications is incorporated by reference in its entirety.

### TECHNICAL FIELD

The present invention relates to methods, controllers, atmosphere control systems and storage units, such as reefer containers and refrigerated trucks and trailers, for transporting produce in an atmosphere-controlled environment, as well as marine vessels comprising such controllers, systems and/or storage units.

### BACKGROUND

Perishable produce can be stored in storage units, such as stationary storage units for storing the produce in a warehouse, or in transportable storage units, also referred to as transport units, for transporting cargo on container vessels. Such a storage unit may comprise an atmosphere control system for controlling an atmosphere in the storage unit. This may be used to facilitate the storage and transportation of perishable goods, such as fruit and vegetables, in the transport unit. Transport units include reefer containers, which may be TEU or 2-TEU containers designed to be shipped on container vessels, and/or refrigerated trucks or trailers.

The storage units may be configured for storing and/or transporting ripenable produce, such as so-called “climacteric” produce, which continues to ripen long after it has been harvested. One example of a climacteric fruit is bananas. Other examples of climacteric produce include avocados, plums, mangos, and other fruits or vegetables. Most respirating produce may be stored and/or transported in this way.

### SUMMARY

A first aspect of the present invention provides a method for maintaining produce in an unripened state in a storage unit, the storage unit comprising a space in which the produce is stored, the method comprising: monitoring a respiration rate of the produce; and in response to a change in the monitored respiration rate, causing an action to be performed to facilitate maintenance of the unripened state.

By monitoring the respiration rate of the produce, a more accurate and direct insight into a state of the produce may be achieved, compared, for example, to monitoring a temperature of the produce. For instance, a change in the respiration rate may precede the produce entering a climacteric state, and so may be indicative of an upcoming change in the ripening state of the produce. Alternatively, or in addition, a change in the respiration rate may be indicative of a temperature hotspot in the container, which could lead to local spoilage of produce. By performing an action to facilitate maintenance of the unripened state in response to a change in the respiration rate, spoilage of the produce, and/or premature ripening of the produce, may be avoided.

This may be particularly advantageous during transport of the produce in a transport unit, such as in a reefer container aboard a marine vessel, where the produce may be in transit for many days or weeks, and where premature initiation of a climacteric state may lead to the produce arriving at a destination in an undesired stage of ripeness, or in a spoiled condition.

The respiration rate may be continuously monitored, or continuously monitored over a predetermined period of time, or may be intermittently monitored. Continuously monitoring the respiration rate over a period of time may enable action to be taken sooner in response to a change in the respiration rate during that period, for instance to delay or reduce a likelihood of the produce entering the climacteric state.

Optionally, monitoring the respiration rate comprises monitoring the output of a gas sensor located in the space, such as a CO2 sensor and/or an O2 sensor. Optionally the respiration rate is derived from the output of the gas sensor.

Optionally, the produce comprises climacteric produce. Optionally, the unripened state comprises a pre-climacteric state. Optionally, the method is a method for maintaining the climacteric produce in a pre-climacteric state. Optionally, the causing the action to be performed is to facilitate maintenance of the pre-climacteric state. Optionally, the storage unit is a ripening unit, such as for use in a ripening warehouse. Optionally, the storage unit is a transport unit. Optionally, the transport unit is a reefer container or refrigerated truck or trailer.

Optionally, the monitored respiration rate comprises any one or more of: an instantaneous respiration rate of the produce; a rate of change of a respiration rate of the produce; a time averaged respiration rate of the produce; and an accumulated respiration rate of the produce over a predetermined period of time.

A change in any one of these parameters, such as a sudden increase in the respiration rate (e.g. as signified by an increase in the rate of change of the respiration rate), or an increase in the accumulated respiration rate compared to that of an earlier time period, may be indicative of an upcoming change in the climacteric state of a climacteric produce. In this way, checking whether the monitored respiration rate has changed may allow remedial action to be taken to avoid, or delay, the produce ripening and/or entering the climacteric state.

Optionally, the causing the action to be performed is in response to the monitored respiration rate exceeding a threshold.

An increase in the monitored respiration rate over the threshold, such as due to an increase in a current or a recent respiration rate, or a sudden increase in respiration rate of the produce, may be indicative of a forthcoming transition of the produce to a ripening or climacteric state. Such a transition may be avoided, or delayed, by determining whether the respiration rate exceeds the threshold and taking action accordingly.

Optionally, the threshold comprises any one or more of: a predetermined value of the monitored respiration rate; and a threshold deviation from a time average value of the monitored respiration rate.

Optionally, the threshold deviation is a predetermined deviation. Optionally, the deviation is a standard deviation, such as two or three standard deviations from the average respiration rate. Optionally, where the monitored respiration rate is a rate of change in respiration rate, the predetermined value or threshold deviation may be represented as an acceptable change in respiration rate over a given time period. Optionally, where the monitored respiration rate is an accumulated respiration rate, the predetermined value or threshold deviation may be represented as an acceptable accumulated respiration rate over a given time period.

Optionally, the method comprises determining the type and/or quantity of the produce in the storage unit, such as by checking a bill of laden for the storage unit. This may be particularly advantageous where the storage unit is a transport unit. Optionally, the predetermined value and/or the threshold deviation is determined based on the type and/or quantity of the produce in the storage unit. Optionally, the method comprises setting the threshold based on the determined type and/or quantity of the produce in the storage unit.

By using a threshold that is a deviation from an average actual respiration rate, or by using a threshold associated with the produce, a change in the respiration rate exceeding the threshold is tailored to the specific produce in the storage unit. This may reduce the likelihood of remedial action being taken unnecessarily, such as where the change of the respiration rate is outside a general threshold for produce, but within expected parameters for the specific produce or for the specific quantity of the produce in the storage unit.

Optionally, the method comprises generating a control signal based on the change in monitored respiration rate, and causing the action to be performed using the control signal.

Optionally, the action comprises any one or more of: issuing an alarm; increasing a flow rate of gas within the space; reducing a temperature in the space; reducing an amount of oxygen in the space; increasing an amount of CO2 in the space; reducing an amount of ethylene in the space; and increasing an amount of ethylene blocker in the space.

Issuing an alarm may enable action to be taken by a person, such as service personnel, to maintain the pre-climacteric state. Alternatively, or in addition, the method may comprise taking action, automatically or otherwise, to maintain the unripened state. Optionally, the method comprises performing any one of the above actions in response to issuance of the alarm.

Increasing a flow rate of gas within the space and/or reducing a temperature in the space may improve a level of cooling of the produce in the space, thereby inhibiting the production of CO2 and delaying, or preventing, the ripening or climacteric state being entered. It may also assist in providing a more even distribution of heat in the space, such as to reduce hotspots and a likelihood of local spoilage.

Similarly, the presence of oxygen and/or ethylene in the space may support the ripening of produce and/or may cause the produce to enter the climacteric state if left uncontrolled. Reducing the amount of oxygen and/or ethylene in the space, and/or increasing an amount of ethylene blocker, such as 1-MCP, in the space, and/or increasing an amount of carbon dioxide in the space, may inhibit ripening of the produce and/or inhibit or delay a transition of the produce within the climacteric state.

Optionally the method further comprises, in response to the change in the respiration rate, causing one or more of: modification of a delivery parameter; and forecasting of a cargo claim.

The delivery parameter may be a time until delivery of the produce to, or collection of the produce by, a recipient of the produce. Where the storage unit is a transport unit, the delivery parameter may be a time until deliver of the transport unit, such as an order in which the transport unit is loaded on to or removed from a marine vessel, or a time when the transport unit is due to leave a storage facility. A change, such as an increase, in the respiration rate may be indicative of a start of ripening of some or all of the produce, and/or a failure of the storage unit to maintain the unripened state. Delivering the produce, such as by shipping the transport unit earlier, and/or redirecting the produce to a difference recipient, may improve a likelihood of the produce arriving at its destination close to a desired state/ripeness. The delivery parameter may be modified in response to the causing the action to issue the alarm.

As noted above, a change, such as an increase, in the respiration rate may be indicative of spoilage or undesired ripening of the produce in the storage unit. The change in the respiration rate may therefore be used to forecast, such as by using a statistical analysis, the likelihood of a cargo claim by a recipient of the produce.

Optionally, the space is couplable to an atmosphere control system that is operable to move gas in the space. Optionally, the method comprises: moving gas in the space at a first flow rate and measuring a first respiration rate of the produce; moving gas in the space at a second flow rate, greater than the first flow rate, and measuring a second respiration rate of the produce; determining a calibration factor based on the measured first and second respiration rates; and calibrating the monitored respiration rate using the in calibration factor. In other words, the change in the monitored respiration rate is a change in the calibrated monitored respiration rate. Optionally, the monitored respiration rate is the first or the second respiration rate. Optionally, the monitored respiration rate is any other monitored respiration rate, such as a respiration rate monitored when gas is moved in the space at any other suitable flow rate.

Moving gas in the space at the second flow rate may provide a pressure difference between the space and an exterior of the storage unit. As such, there may be a detectable exchange of gas into or out of the space through gaps in the storage unit, such as in proximity to a door, a refrigerant conduit, a ventilation port, or other part of the storage unit, when the gas is moved at the second flow rate.

Optionally, the first flow rate is zero, or close to zero. That is, the gas in the space may be substantially stationary. Optionally, the first flow rate is such that a pressure difference between the space and an exterior of the storage unit is zero, or close to zero when the gas is moved in the space at the first flow rate. That is, there may be minimal exchange of gas from the space to the exterior, and/or from the exterior to the space, when the gas is moved in the space at the first flow rate. In this way, the calibration factor may provide in indication of an amount of leakage of gas into and/or out of the space when the gas is moved in the space at the second flow rate.

Alternatively, the first flow rate is any other flow rate less than the second flow rate. For example, moving the gas in the space at the first flow rate may comprise operating the gas moving device at or around half its maximum speed, and moving the gas in the space at the second flow rate may comprise operating the gas moving device at or around its maximum speed. Operating the gas moving device at its maximum speed may provide a pressure difference that is up to twice as large, up to 3 times as large, up to 5 times as large, or more than 5 times as large as the pressure difference when operating the gas moving device at half its maximum speed. Optionally, the maximum speed of the fan is up to 1750 rpm, up to 3500 rpm, or over 3500 rpm. Optionally, the first and second flow rates may be any other suitable flow rates such as to provide different pressure differences between an internal location in the storage unit and the exterior of the storage unit, wherein the difference between pressure differences is large enough to allow the calibration factor to be determined.

Optionally, the atmosphere control system comprises a gas moving device, such as a fan, and the first and second flow rates are provided by operating the fan at respective first and second speeds, such as first and second rotational speeds. Optionally, the gas moving device is located in the space. Optionally, the storage unit comprises at least one port through which gas is supplied from the atmosphere control system, such as from the gas moving device, to the space. Optionally, the first and second flow rates are first and second flow rates of gas supplied to the space. In other words, the storage unit may be couplable to the atmosphere control system via the at least one port. Optionally, the moving gas in the space at the first flow rate comprises restricting and/or preventing a flow of gas through the at least one port.

Optionally, when the first flow rate is zero, no gas is supplied to the space through the at least one port. This may be by closing the at least one port, or by uncoupling the atmosphere control system and the space. For instance, the atmosphere control system may be separate to the storage unit, such as an atmosphere control system for supplying gas to plural storage units, and the space may be uncoupled from the atmosphere control system when the storage unit is being moved, such as when it is being loaded onto a container ship. Alternatively, or in addition, the atmosphere control system may be uncoupled from the space during a defrost mode of the atmosphere control system, in which the gas moving device may be operable to defrost a heat exchanger of the atmosphere control system. In this case, the at least one port may be at least partially closed to limit or prevent a movement of gas into the space to achieve the first flow rate.

The first and second respiration rates may be determined in any order. For instance, the method may comprise determining the first respiration rate when moving the gas at the first flow rate before determining the second respiration rate when moving the gas at the second flow rate, or may alternatively comprise determining the second respiration rate when moving the gas at the second flow rate before determining the first respiration rate when moving the gas at the first flow rate.

Optionally, the calibration factor is determined based on a ratio, or difference, between the first and second respiration rates. Optionally, the method comprises determining the ratio, or difference, between the first and second respiration rates.

Optionally, the method comprises storing the calibration factor and/or the calibrated monitored respiration rate, such as in a computer-readable memory. Optionally, the method comprises transmitting a signal indicative of the calibration factor and/or the calibrated monitored respiration rate, to the storage unit and/or to the atmosphere control system.

Optionally, the method comprises measuring the first and/or second respiration rate using only one type of gas sensor. The gas sensor may be a CO2 sensor. That is, the first and/or second respiration rate may be measured without using an O2 sensor.

The calibrated monitored respiration rate may provide a more accurate indication of the actual respiration rate of the produce, including accounting for any leakage of gas into and/or out of the container when the gas is moved in the space. This may be particularly advantageous where only a single type of gas sensor, such as a CO2 sensor, is used to measure the first and/or the second respiration rate. That is, a more accurate respiration rate may be measured without also using an O2 sensor to determine a leakage rate. This may reduce a cost of the storage unit, increase a longevity of the storage unit, and/or improve an ease of maintenance of the storage unit.

Optionally, the calibrated monitored respiration rate is determined based on a speed correlation factor. This may allow a more accurate determination of the calibrated monitored respiration rate, which may account for changing pressure differences and/or leakage rates as the flow rate of gas in the space is varied.

Optionally, the speed correlation factor is predetermined. Optionally, the speed correlation factor is determined based on two or more calibration factors, such as previously-determined calibration factors, which may each be associated with a respective gas flow rate in the space. Optionally, the method comprises determining the speed correlation factor.

Optionally, the first and second respiration rates are measured at respective first and second times, and the method further comprises: at a third time, after the first and second times, moving gas in the space at a third flow rate and measuring a third respiration rate; and updating the calibration factor based on the third respiration rate.

Optionally, the first time is before the second time, the third flow rate is less than the second flow rate, and the calibration factor is updated based on the third respiration rate and the second respiration rate. In this case, optionally, the third flow rate is the same as the first flow rate. Alternatively, the third flow rate may be greater than, or less than, the first flow rate. Optionally, the third flow rate is zero, or close to zero, so that a pressure difference between an interior and an exterior of the space is reduced or minimised.

Alternatively, the first time is after the second time, the third flow rate is greater than the first flow rate, and the calibration factor is updated based on the first respiration rate and the third respiration rate. In this case, optionally, the third flow rate is the same as the second flow rate. Alternatively, the third flow rate may be greater than, or less than, the second flow rate. Optionally, in this case, moving the gas in the space at the third flow rate may provide a pressure difference between the space and an exterior of the storage unit.

Optionally, the method comprises, at a fourth time, moving gas in the space at a fourth flow rate, different to the third flow rate, and measuring a fourth respiration rate. Optionally, the method comprises updating the calibration factor based on the third respiration rate and the fourth respiration rate. Optionally, both the third and fourth times are after the first and second times. Optionally, the third time is before the fourth time. Alternatively, the third time may be after the fourth time.

Optionally, the higher of the third and fourth flow rates is the same as, greater than, or less than the second flow rate. Optionally, the lower of the third and fourth flow rates is the same as, greater than, or lower than the first flow rate. Optionally, the lower of the third and fourth flow rates is zero, or close to zero. Optionally, the lower of the third and fourth flow rates is such that a pressure difference between the space and an exterior of the storage unit is zero, or close to zero when the gas is moved in the space at the lower of the third and fourth flow rates. Optionally, moving the gas in the space at the higher of the third and fourth flow rates may provide a pressure difference between the space and an exterior of the storage unit.

In other words, generally speaking, the calibration factor may be updated by changing a flow rate of gas in the space from a high flow rate to a low flow rate, or vice versa, and determining a calibration factor based on respiration rates measured at the respective high and low flow rates. The high and low flow rates may be “high” and “low” relative to each other, so that at the high flow rate a pressure difference may be present between at least a part of the space and an external atmosphere, and at the low flow rate, such a pressure difference is low, such as minimal, or negligible.

Optionally, the updating the calibration factor comprises determining a new calibration factor based on any suitable combination of measured respiration rates, and modifying, such as adjusting, the calibration factor based on the new calibration factor. Optionally, the updating the calibration factor comprises replacing the calibration factor with the new calibration factor.

Optionally, the method comprises determining the calibration factor when one or more predetermined conditions have been met, the one or more predetermined conditions being any one or more of: the storage unit being loaded onto a container ship; the atmosphere control system or a part thereof being inoperable, or uncoupled from the space; an atmosphere in the space reaching a predetermined temperature; an atmosphere in the space reaching a predetermined composition; an atmosphere in the space being stable; the passage of a predetermined period of time since the calibration factor was last determined; and a change in an external temperature and/or pressure reaching a predetermined threshold.

Optionally, the method comprises updating the calibration factor in any suitable way as described above when the one or more predetermined conditions is met.

The storage unit may be a reefer container. The storage unit may comprise the atmosphere control system coupled to the space, the atmosphere control system comprising a gas moving device, such as a fan, for causing gas to be moved in the space. The gas moving device may be inoperable, or may run slowly, during loading of the reefer container onto the container ship.

A stable atmosphere may mean that a temperature and/or composition of the atmosphere has remained relatively unchanged for a predetermined period of time.

The calibration factor may be determined, and/or updated, at predetermined intervals of time, such as to provide an up-to-date indication of a leakage of gas into and/or out of the unit, and/or to provide an indication of an amount of leakage as a function of a flow rate of gas in the space, or information representative thereof, such as a speed of the gas moving device. The interval of time may be up to 15 minutes, up to 30 minutes, up to one hour, up to 2 hours, up to 12 hours, up to 1 day, or more than 1 day. That is, the calibration factor may be updated once, or a couple of times during a transport event of the storage unit. The respiration rate may be determined, such as based on the calibration factor, once in a number of hours less than 4, up to once every 4 hours, up to once every 8 hours, up to once every 10 hours, or once in a number of hours greater than 10.

The predetermined temperature and/or composition being reached may permit the gas to be moved in the space at a reduced flow rate for a period of time to measure the second respiration rate, or other lower respiration rate, without compromising an integrity of the cargo. For instance, the cargo may be produce, and ensuring the atmosphere is at or below a predetermined set temperature, or has a set composition, may permit the gas moving device to operate more slowly, such as to be inoperable, for a period of time without causing spoilage and/or premature ripening of the produce, such as due to an increase in temperature and/or change in composition of the atmosphere in the space.

A change in an external temperature and/or pressure may affect a pressure difference between the space and an external atmosphere, and/or leakage rate of the gas into and/or out of the space. This may prompt an update to the calibration factor, such as to improve an accuracy of respiration rates measured following the change in external temperature and/or pressure.

A second aspect of the present invention provides a controller configured to perform the method of the first aspect.

The controller may be configured to perform the action itself, or may be configured to cause another system to perform the action. The controller may be in the storage unit, or may be a remote controller, such as a controller of a marine vessel on which the storage unit is located, or a cloud-based controller that is communicatively coupled to the storage unit and/or the marine vessel. The controller may comprise a plurality of controllers, each of the plurality of controllers configured to perform one or more operations of the method of the first aspect.

A third aspect of the present invention provides a non-transitory computer-readable storage medium storing instructions that, when executed by a processor of a controller, such as the controller of the second aspect, cause the processor to perform the method of the first aspect.

A fourth aspect of the present invention provides an atmosphere control system operable by the controller of the second aspect, the atmosphere control system configured to control an atmosphere in the space of the storage unit, and to perform the action to facilitate maintenance of the unripened state of the produce.

Optionally, the produce is climacteric produce. Optionally, the unripened state comprises a pre-climacteric state. Optionally, the action is to facilitate maintenance of the pre-climacteric state.

Optionally, the action is any one or more of: issuing an alarm; increasing a flow rate of gas within the space; reducing a temperature in the space; reducing an amount of oxygen in the space; increasing an amount of carbon dioxide in the space; reducing an amount of ethylene in the space; and increasing an amount of ethylene blocker in the space.

The atmosphere control system may comprise the controller or the controller may be a remote controller, such as a controller of a marine vessel on which the atmosphere control system is located, or a cloud-based controller that is communicatively coupled to the storage unit and/or the marine vessel. Optionally, the atmosphere control system is controlled by the controller, and/or receives signals from the controller which cause the atmosphere control system to perform the action to facilitate maintenance of the climacteric state. Optionally, the atmosphere control system is comprised in the storage unit. Optionally, the atmosphere control system is configured to control an atmosphere in more than one storage unit.

Optionally, the storage unit is a ripening unit, such as for use in a ripening warehouse. Optionally, the storage unit is a transport unit. Optionally, the transport unit is a reefer container or refrigerated truck or trailer.

Optionally, the atmosphere control system comprises a fan for controlling an amount of gas moved in, and/or supplied to the space. Optionally, the increasing a flow of gas within the space comprises increasing a speed of the fan.

Optionally, the atmosphere control system comprises a heat exchanger configured to adjust a temperature of gas supplied to the space, and the reducing the temperature in the space comprises reducing a temperature setpoint of the heat exchanger.

Optionally, the atmosphere control system comprises a composition adjuster configured to adjust, or change, a composition of gas supplied to the space. Optionally, the reducing an amount of oxygen and/or ethylene in the space comprises operating the composition adjuster to remove oxygen and/or ethylene from the gas supplied to the space. The composition adjuster may comprise an oxygen and/or ethylene scrubber configured to remove oxygen and/or ethylene from return gas received by the atmosphere control system from the space.

Optionally, the composition adjuster comprises an ethylene-blocker injector, and/or carbon dioxide injector, respectively configured to increase an amount of an ethylene blocker, such as 1-MCP, and/or carbon dioxide in the gas supplied to the space. Optionally, the increasing the amount of ethylene blocker and/or carbon dioxide in the space comprises operating the composition adjuster to increase the amount of ethylene blocker and/or carbon dioxide in the space respectively.

A fifth aspect of the present invention provides a storage unit comprising, or couplable to, the atmosphere control system of the fourth aspect, the storage unit comprising the space in which the produce is stored.

Optionally, the storage unit comprises the controller of the second aspect, and/or the non-transitory computer-readable storage medium of the third aspect.

Optionally, the storage unit is a ripening unit, such as for use in a ripening warehouse. Optionally, the storage unit is a transport unit. Optionally, the transport unit is a reefer container or refrigerated truck or trailer.

A sixth aspect of the present invention provides a marine vessel comprising the controller of the second aspect, the atmosphere control system of the fourth aspect, or the storage unit of the fifth aspect.

A seventh aspect of the present invention provides a method of determining a respiration rate of produce in a storage unit, the storage unit comprising a space in which the produce is stored, the space being couplable to an atmosphere control system that is operable to move gas in the space, the method comprising: moving gas in the space at a first flow rate and measuring a first respiration rate of the produce; moving gas in the space at a second flow rate, greater than the first flow rate, and measuring a second respiration rate of the produce; determining a calibration factor based on the measured first and second respiration rates; and determining a calibrated respiration rate based on the calibration factor.

Moving gas in the space at the second flow rate may provide a pressure difference between the space and an exterior of the storage unit. As such, there may be a detectable exchange of gas into or out of the space through gaps in the storage unit, such as in proximity to a door, a refrigerant conduit, a ventilation port, or other part of the storage unit, when the gas is moved at the second flow rate.

Optionally, the first flow rate is zero, or close to zero. That is, the gas in the space may be substantially stationary. Optionally, the first flow rate is such that a pressure difference between the space and an exterior of the storage unit is zero, or close to zero when the gas is moved in the space at the first flow rate. That is, there may be minimal exchange of gas from the space to the exterior, and/or from the exterior to the space, when the gas is moved in the space at the first flow rate. In this way, the calibration factor may provide in indication of an amount of leakage of gas into and/or out of the space when the gas is moved in the space at the second flow rate.

Alternatively, the first flow rate is any other flow rate less than the second flow rate. For example, moving the gas in the space at the first flow rate may comprise operating the gas moving device at or around half its maximum speed, and moving the gas in the space at the second flow rate may comprise operating the gas moving device at or around its maximum speed. Operating the gas moving device at its maximum speed may provide a pressure difference that is up to twice as large, up to 3 times as large, up to 5 times as large, or more than 5 times as large as the pressure difference when operating the gas moving device at half its maximum speed. Optionally, the maximum speed of the fan is up to 1750 rpm, up to 3500 rpm, or over 3500 rpm. Optionally, the first and second flow rates may be any other suitable flow rates such as to provide different pressure differences between an internal location in the storage unit and the exterior of the storage unit, wherein the difference between pressure differences is large enough to allow the calibration factor to be determined.

Optionally, the atmosphere control system comprises a gas moving device, such as a fan, and the first and second flow rates are provided by operating the fan at respective first and second speeds, such as first and second rotational speeds. Optionally, the gas moving device is located in the space. Optionally, the storage unit comprises at least one port through which gas is supplied from the atmosphere control system, such as from the gas moving device, to the space. Optionally, the first and second flow rates are first and second flow rates of gas supplied to the space. In other words, the storage unit may be couplable to the atmosphere control system via the at least one port. Optionally, the moving gas in the space at the first flow rate comprises restricting and/or preventing a flow of gas through the at least one port.

Optionally, when the first flow rate is zero, no gas is supplied to the space through the at least one port. This may be by closing the at least one port, or by uncoupling the atmosphere control system and the space. For instance, the atmosphere control system may be separate to the storage unit, such as an atmosphere control system for supplying gas to plural storage units, and the space may be uncoupled from the atmosphere control system when the storage unit is being moved, such as when it is being loaded onto a container ship. Alternatively, or in addition, the atmosphere control system may be uncoupled from the space during a defrost mode of the atmosphere control system, in which the gas moving device may be operable to defrost a heat exchanger of the atmosphere control system. In this case, the at least one port may be at least partially closed to limit or prevent a movement of gas into the space to achieve the first flow rate.

The first and second respiration rates may be determined in any order. For instance, the method may comprise determining the first respiration rate when moving the gas at the first flow rate before determining the second respiration rate when moving the gas at the second flow rate, or may alternatively comprise determining the second respiration rate when moving the gas at the second flow rate before determining the first respiration rate when moving the gas at the first flow rate.

Optionally, the calibration factor is determined based on a ratio, or difference, between the first and second respiration rates. Optionally, the method comprises determining the ratio, or difference, between the first and second respiration rates.

Optionally, the method comprises storing the calibration factor and/or the calibrated respiration rate, such as in a computer-readable memory. Optionally, the method comprises transmitting a signal indicative of the calibration factor and/or the calibrated respiration rate, to the storage unit and/or to the atmosphere control system.

Optionally, the method comprises measuring the first and/or second respiration rate using only one type of gas sensor. The gas sensor may be a CO2 sensor. That is, the first and/or second respiration rate may be measured without using an O2 sensor.

Optionally, the calibrated respiration rate is determined based on the calibration factor and a respiration rate to be calibrated. The calibrated respiration rate may provide a more accurate indication of the actual respiration rate of the produce, including accounting for any leakage of gas into and/or out of the container when the gas is moved in the space. This may be particularly advantageous where only a single type of gas sensor, such as a CO2 sensor, is used to measure the first and/or the second respiration rate. That is, a more accurate respiration rate may be measured without also using an O2 sensor to determine a leakage rate. This may reduce a cost of the storage unit, increase a longevity of the storage unit, and/or improve an ease of maintenance of the storage unit.

Optionally, the respiration rate to be calibrated is the first or second respiration rate. Optionally, the method comprises measuring a further respiration rate, such as when gas is moved in the compartment at a further flow rate, which may be the same as, greater than, or less than the first or the second flow rate. Optionally, the respiration rate to be calibrated is the measured further respiration rate.

Optionally, the method comprises multiplying the respiration rate to be calibrated by the calibration factor to obtain the calibrated respiration rate.

Optionally, the calibrated respiration rate is determined based on a speed correlation factor. This may allow a more accurate determination of the calibrated respiration rate, which may account for changing pressure differences and/or leakage rates as the flow rate of gas in the space is varied.

Optionally, the speed correlation factor is predetermined. Optionally, the speed correlation factor is determined based on two or more calibration factors, such as previously-determined calibration factors, which may each be associated with a respective gas flow rate in the space. Optionally, the method comprises determining the speed correlation factor.

Optionally, the calibration factor is used to determine plural calibrated respiration rates over time. Optionally, the respiration rate to be calibrated is continually or intermittently monitored, and the calibrated respiration rate is correspondingly continually, or intermittently, determined using the calibration factor and/or the speed correlation factor.

Optionally, the first and second respiration rates are measured at respective first and second times, and the method further comprises: at a third time, after the first and second times, moving gas in the space at a third flow rate and measuring a third respiration rate; and updating the calibration factor based on the third respiration rate.

Optionally, the first time is before the second time, the third flow rate is less than the second flow rate, and the calibration factor is updated based on the third respiration rate and the second respiration rate. In this case, optionally, the third flow rate is the same as the first flow rate. Alternatively, the third flow rate may be greater than, or less than, the first flow rate. Optionally, the third flow rate is zero, or close to zero, so that a pressure difference between an interior and an exterior of the space is reduced or minimised.

Alternatively, the first time is after the second time, the third flow rate is greater than the first flow rate, and the calibration factor is updated based on the first respiration rate and the third respiration rate. In this case, optionally, the third flow rate is the same as the second flow rate. Alternatively, the third flow rate may be greater than, or less than, the second flow rate. Optionally, in this case, moving the gas in the space at the third flow rate may provide a pressure difference between the space and an exterior of the storage unit.

Optionally, the method comprises, at a fourth time, moving gas in the space at a fourth flow rate, different to the third flow rate, and measuring a fourth respiration rate. Optionally, the method comprises updating the calibration factor based on the third respiration rate and the fourth respiration rate. Optionally, both the third and fourth times are after the first and second times. Optionally, the third time is before the fourth time. Alternatively, the third time may be after the fourth time.

Optionally, the higher of the third and fourth flow rates is the same as, greater than, or less than the second flow rate. Optionally, the lower of the third and fourth flow rates is the same as, greater than, or lower than the first flow rate. Optionally, the lower of the third and fourth flow rates is zero, or close to zero. Optionally, the lower of the third and fourth flow rates is such that a pressure difference between the space and an exterior of the storage unit is zero, or close to zero when the gas is moved in the space at the lower of the third and fourth flow rates. Optionally, moving the gas in the space at the higher of the third and fourth flow rates may provide a pressure difference between the space and an exterior of the storage unit.

In other words, generally speaking, the calibration factor may be updated by changing a flow rate of gas in the space from a high flow rate to a low flow rate, or vice versa, and determining a calibration factor based on respiration rates measured at the respective high and low flow rates. The high and low flow rates may be “high” and “low” relative to each other, so that at the high flow rate a pressure difference may be present between at least a part of the space and an external atmosphere, and at the low flow rate, such a pressure difference is minimal, or negligible.

Optionally, the updating the calibration factor comprises determining a new calibration factor based on any suitable combination of measured respiration rates. and modifying, such as adjusting, the calibration factor based on the new calibration factor. Optionally, the updating the calibration factor comprises replacing the calibration factor with the new calibration factor.

Optionally, the method comprises determining the calibration factor when one or more predetermined conditions have been met, the one or more predetermined conditions being any one or more of: the storage unit being loaded onto a container ship; the atmosphere control system or a part thereof being inoperable, or uncoupled from the space; an atmosphere in the space reaching a predetermined temperature; an atmosphere in the space reaching a predetermined composition; an atmosphere in the space being stable; the passage of a predetermined period of time since the calibration factor was last determined; and a change in an external temperature and/or pressure reaching a predetermined threshold.

Optionally, the method comprises updating the calibration factor in any suitable way as described above when the one or more predetermined conditions is met.

The storage unit may be a reefer container. The storage unit may comprise the atmosphere control system coupled to the space, the atmosphere control system comprising a gas moving device, such as a fan, for causing gas to be moved in the space. The gas moving device may be inoperable, or may run slowly, during loading of the reefer container onto the container ship.

A stable atmosphere may mean that a temperature and/or composition of the atmosphere has remained relatively unchanged for a predetermined period of time.

The calibration factor may be determined, and/or updated, at predetermined intervals of time, such as to provide an up-to-date indication of a leakage of gas into and/or out of the unit, and/or to provide an indication of an amount of leakage as a function of a flow rate of gas in the space, or information representative thereof, such as a speed of the gas moving device. The interval of time may be up to 15 minutes, up to 30 minutes, up to one hour, up to 2 hours, up to 12 hours, up to 1 day, or more than 1 day. That is, the calibration factor may be updated once, or a couple of times during a transport event of the transport unit. The respiration rate may be determined, such as based on the calibration factor, once in a number of hours less than 4, up to once every 4 hours, up to once every 8 hours, up to once every 10 hours, or once in a number of hours greater than 10.

The predetermined temperature and/or composition being reached may permit the gas to be moved in the space at a reduced flow rate for a period of time to measure the second respiration rate, or other lower respiration rate, without compromising an integrity of the cargo. For instance, the cargo may be produce, and ensuring the atmosphere is at or below a predetermined set temperature, or has a set composition, may permit the gas moving device to operate more slowly, such as to be inoperable, for a period of time without causing spoilage and/or premature ripening of the produce, such as due to an increase in temperature and/or change in composition of the atmosphere in the space.

A change in an external temperature and/or pressure may affect a pressure difference between the space and an external atmosphere, and/or leakage rate of the gas into and/or out of the space. This may prompt an update to the calibration factor, such as to improve an accuracy of respiration rates measured following the change in external temperature and/or pressure.

An eighth aspect provides a method of controlling a ripening process of ripenable produce, the method comprising determining a calibrated respiration rate in accordance with the seventh, aspect and controlling the ripening process based on the calibrated respiration rate. Optionally, the produce is climacteric produce, and the method comprises adjusting a rate of ripening of the produce when the produce is in a climacteric state, such as by changing a temperature and/or composition of an atmosphere surrounding the produce in response to a change in the calibrated respiration rate. By using a calibrated respiration rate, the ripening process may be more accurately controlled.

A ninth aspect provides a method of maintaining produce in an unripened state in a storage unit, the method comprising monitoring a calibrated respiration rate of the produce, the calibrated respiration rate determined in accordance with the seventh aspect, and, in response to a change in the calibrated monitored respiration rate, causing an action to be performed to facilitate maintenance of the unripened state.

By monitoring the calibrated respiration rate of the produce, a more accurate and direct insight into a state of the produce may be achieved, compared, for example, to monitoring a temperature of the produce. For instance, a change in the calibrated respiration rate may precede the produce entering a climacteric state, and so may be indicative of an upcoming change in the ripening state of the produce. Alternatively, or in addition, a change in the calibrated respiration rate may be indicative of a temperature hotspot in the container, which could lead to local spoilage of produce. By performing an action to facilitate maintenance of the unripened state in response to a change in the calibrated respiration rate, spoilage of the produce, and/or premature ripening of the produce, may be avoided.

This may be particularly advantageous during transport of the produce in a transport unit, such as in a reefer container aboard a marine vessel, where the produce may be in transit for many days or weeks, and where premature initiation of a climacteric state may lead to the produce arriving at a destination in an undesired stage of ripeness, or in a spoiled condition.

The calibrated respiration rate may be continuously monitored, or continuously monitored over a predetermined period of time, or may be intermittently monitored. Continuously monitoring the calibrated respiration rate over a period of time may enable action to be taken sooner in response to a change in the calibrated respiration rate during that period, for instance to delay or reduce a likelihood of the produce entering the climacteric state.

Optionally, monitoring the calibrated respiration rate comprises monitoring the output of a gas sensor located in the space, such as a CO2 sensor and/or an O2 sensor. Optionally the calibrated respiration rate is derived from the output of the gas sensor.

Optionally, the produce comprises climacteric produce. Optionally, the unripened state comprises a pre-climacteric state. Optionally, the method is a method for maintaining the climacteric produce in a pre-climacteric state. Optionally, the causing the action to be performed is to facilitate maintenance of the pre-climacteric state. Optionally, the storage unit is a ripening unit, such as for use in a ripening warehouse. Optionally, the storage unit is a transport unit. Optionally, the transport unit is a reefer container or refrigerated truck or trailer.

Optionally, the calibrated monitored respiration rate comprises any one or more of: an instantaneous calibrated respiration rate of the produce; a rate of change of a calibrated respiration rate of the produce; a time averaged calibrated respiration rate of the produce; and an accumulated calibrated respiration rate of the produce over a predetermined period of time.

A change in any one of these parameters, such as a sudden increase in the calibrated respiration rate (e.g. as signified by an increase in the rate of change of the calibrated respiration rate), or an increase in the accumulated calibrated respiration rate compared to that of an earlier time period, may be indicative of an upcoming change in the climacteric state of a climacteric produce. In this way, checking whether the calibrated monitored respiration rate has changed may allow remedial action to be taken to avoid, or delay, the produce ripening and/or entering the climacteric state.

Optionally, the causing the action to be performed is in response to the calibrated monitored respiration rate exceeding a threshold.

An increase in the calibrated monitored respiration rate over the threshold, such as due to an increase in a current or a recent respiration rate, or a sudden increase in calibrated respiration rate of the produce, may be indicative of a forthcoming transition of the produce to a ripening or climacteric state. Such a transition may be avoided, or delayed, by determining whether the respiration rate exceeds the threshold and taking action accordingly.

Optionally, the threshold comprises any one or more of: a predetermined value of the calibrated monitored respiration rate; and a threshold deviation from a time average value of the calibrated monitored respiration rate.

Optionally, the threshold deviation is a predetermined deviation. Optionally, the deviation is a standard deviation, such as two or three standard deviations from the average calibrated respiration rate. Optionally, where the calibrated monitored respiration rate is a rate of change in calibrated respiration rate, the predetermined value or threshold deviation may be represented as an acceptable change in calibrated respiration rate over a given time period. Optionally, where the calibrated monitored respiration rate is an accumulated calibrated respiration rate, the predetermined value or threshold deviation may be represented as an acceptable accumulated calibrated respiration rate over a given time period.

Optionally, the method comprises determining the type and/or quantity of the produce in the storage unit, such as by checking a bill of laden for the storage unit. This may be particularly advantageous where the storage unit is a transport unit. Optionally, the predetermined value and/or the threshold deviation is determined based on the type and/or quantity of the produce in the storage unit. Optionally, the method comprises setting the threshold based on the determined type and/or quantity of the produce in the storage unit.

By using a threshold that is a deviation from an average actual calibrated respiration rate, or by using a threshold associated with the produce, a change in the calibrated respiration rate exceeding the threshold is tailored to the specific produce in the storage unit. This may reduce the likelihood of remedial action being taken unnecessarily, such as where the change of the calibrated respiration rate is outside a general threshold for produce, but within expected parameters for the specific produce or for the specific quantity of the produce in the storage unit.

Optionally, the action comprises any one or more of: issuing an alarm; increasing a flow rate of gas within the space; reducing a temperature in the space; reducing an amount of oxygen in the space; increasing an amount of carbon dioxide in the space; reducing an amount of ethylene in the space; and increasing an amount of ethylene blocker in the space.

Issuing an alarm may enable action to be taken by a person, such as service personnel, to maintain the pre-climacteric state. Alternatively, or in addition, the method may comprise taking action, automatically or otherwise, to maintain the unripened state.

Optionally, the method comprises performing any one of the above actions in response to issuance of the alarm.

Increasing a flow rate of gas within the space and/or reducing a temperature in the space may improve a level of cooling of the produce in the space, thereby inhibiting the production of CO2 and delaying, or preventing, the ripening or climacteric state being entered. It may also assist in providing a more even distribution of heat in the space, such as to reduce hotspots and a likelihood of local spoilage.

Similarly, the presence of oxygen and/or ethylene in the space may support the ripening of produce and/or may cause the produce to enter the climacteric state if left uncontrolled. Reducing the amount of oxygen and/or ethylene in the space, and/or increasing an amount of ethylene blocker, such as 1-MCP, in the space, and/or increasing an amount of carbon dioxide in the space, may inhibit ripening of the produce and/or inhibit or delay a transition of the produce within the climacteric state.

Optionally the method further comprises, in response to the change in the calibrated respiration rate, causing one or more of: modification of a delivery parameter; and forecasting of a cargo claim.

The delivery parameter may be a time until delivery of the produce to, or collection of the produce by, a recipient of the produce. Where the storage unit is a transport unit, the delivery parameter may be a time until deliver of the transport unit, such as an order in which the transport unit is loaded on to or removed from a marine vessel, or a time when the transport unit is due to leave a storage facility. A change, such as an increase, in the calibrated respiration rate may be indicative of a start of ripening of some or all of the produce, and/or a failure of the storage unit to maintain the unripened state. Delivering the produce, such as by shipping the transport unit earlier, or redirecting the produce to a different recipient, may improve a likelihood of the produce arriving at its destination close to a desired state/ripeness. The delivery parameter may be modified in response to the causing the action to issue the alarm

As noted above, a change, such as an increase, in the calibrated respiration rate may be indicative of spoilage or undesired ripening of the produce in the storage unit. The change in the calibrated respiration rate may therefore be used to forecast, such as by using a statistical analysis, the likelihood of a cargo claim by a recipient of the produce.

A tenth aspect of the present invention provides a controller configured to perform the method of the any one of the seventh to ninth aspects.

The controller may be configured to perform the action itself, or may be configured to cause another system to perform the action. The controller may be in the storage unit, or may be a remote controller, such as a controller of a marine vessel on which the storage unit is located, or a cloud-based controller that is communicatively coupled to the storage unit and/or the marine vessel. The controller may comprise a plurality of controllers, each of the plurality of controllers configured to perform one or more operations of the method of the first aspect.

An eleventh aspect of the present invention provides a non-transitory computer-readable storage medium storing instructions that, when executed by a processor of a controller, such as the controller of the tenth aspect, cause the processor to perform the method of any one of the seventh to ninth aspects.

A twelfth aspect of the present invention provides an atmosphere control system operable by the controller of the tenth aspect, the atmosphere control system configured to control an atmosphere in the space of the storage unit. Optionally, the atmosphere control system is configured to perform the action to facilitate maintenance of the unripened state of the produce.

Optionally, the produce is climacteric produce. Optionally, the unripened state comprises a pre-climacteric state. Optionally, the action is to facilitate maintenance of the pre-climacteric state.

Optionally, the action is any one or more of: issuing an alarm; increasing a flow rate of gas within the space; reducing a temperature in the space; reducing an amount of oxygen in the space; increasing an amount of CO2 in the space; reducing an amount of ethylene in the space; and increasing an amount of ethylene blocker in the space.

The atmosphere control system may comprise the controller or the controller may be a remote controller, such as a controller of a marine vessel on which the atmosphere control system is located, or a cloud-based controller that is communicatively coupled to the storage unit and/or the marine vessel. Optionally, the atmosphere control system is controlled by the controller, and/or receives signals from the controller which cause the atmosphere control system to perform the action to facilitate maintenance of the climacteric state. Optionally, the atmosphere control system is comprised in the storage unit. Optionally, the atmosphere control system is configured to control an atmosphere in more than one storage unit.

Optionally, the storage unit is a ripening unit, such as for use in a ripening warehouse. Optionally, the storage unit is a transport unit. Optionally, the transport unit is a reefer container or refrigerated truck or trailer.

Optionally, the atmosphere control system comprises a fan for controlling an amount of gas moved in, and/or supplied to the space. Optionally, the increasing a flow of gas within the space comprises increasing a speed of the fan.

Optionally, the atmosphere control system comprises a heat exchanger configured to adjust a temperature of gas supplied to the space, and the reducing the temperature in the space comprises reducing a temperature setpoint of the heat exchanger.

Optionally, the atmosphere control system comprises a composition adjuster configured to adjust, or change, a composition of gas supplied to the space. Optionally, the reducing an amount of oxygen and/or ethylene in the space comprises operating the composition adjuster to remove oxygen and/or ethylene from the gas supplied to the space. The composition adjuster may comprise an oxygen and/or ethylene scrubber configured to remove oxygen and/or ethylene from return gas received by the atmosphere control system from the space.

Optionally, the composition adjuster comprises an ethylene-blocker injector configured to increase an amount of an ethylene blocker, such as 1-MCP, in the gas supplied to the space. Optionally, the increasing the amount of ethylene blocker in the space comprises operating the composition adjuster to increase the amount of ethylene blocker in the space.

Optionally, the composition adjuster comprises a carbon dioxide injector configured to increase an amount of carbon dioxide in the gas supplied to the space. Optionally, the increasing the amount of carbon dioxide in the space comprises operating the composition adjuster to increase the amount of carbon dioxide in the space.

A thirteenth aspect of the present invention provides a storage unit comprising, or couplable to, the atmosphere control system of the twelfth aspect, the storage unit comprising the space in which the produce is stored.

Optionally, the storage unit comprises the controller of the tenth aspect, and/or the non-transitory computer-readable storage medium of the eleventh aspect.

Optionally, the storage unit is a ripening unit, such as for use in a ripening warehouse. Optionally, the storage unit is a transport unit. Optionally, the transport unit is a reefer container or refrigerated truck or trailer.

A fourteenth aspect of the present invention provides a marine vessel comprising the controller of the tenth aspect, the atmosphere control system of the twelfth aspect, or the storage unit of the thirteenth aspect.

It will be appreciated that features of any one of the above aspects may be combined with features of any other of the above aspects. Similarly, any of the optional features for one aspect may be combined those of another aspect. For instance, the monitored respiration rate of the first aspect may be calibrated in any suitable way in accordance with the seventh aspect. Alternatively, or in addition, the calibrated respiration rate of the seventh aspect may be used for maintaining produce in an unripened state in accordance with the first aspect.

### BRIEF DESCRIPTION OF THE DRAWINGS

Embodiments of the invention will now be described, by way of example only, with reference to the accompanying drawings, in which:

FIG. 1 shows a schematic view of a storage unit according to an example;

FIG. 2 show a schematic view of a marine vessel comprising the storage unit of FIG. 1;

FIG. 3 shows an example CO2 production curve of a climacteric produce;

FIG. 4A shows an example method for maintaining produce in an unripened state;

FIG. 4B shows an example method of determining a respiration rate of produce in a storage unit;

FIG. 5 shows an example control system comprising a controller configured to perform the example method of FIG. 4; and

FIG. 6 shows a non-transitory computer-readable storage medium according to an example.

Further features and advantages of the invention will become apparent from the following description of preferred embodiments of the invention, given by way of example only, which is made with reference to the accompanying drawings.

### DETAILED DESCRIPTION

In the following description, examples of the invention are described in relation to a storage unit that is a reefer container. It will be understood that the invention is not limited to this purpose, and may be applied to any kind of storage unit, for example to a refrigerated truck or trailer, or any other type of storage unit, such as a storage unit for use in a ripening warehouse. Further, although examples are described in relation to a so-called “climacteric” produce, the invention is also applicable to any suitable respirating produce.

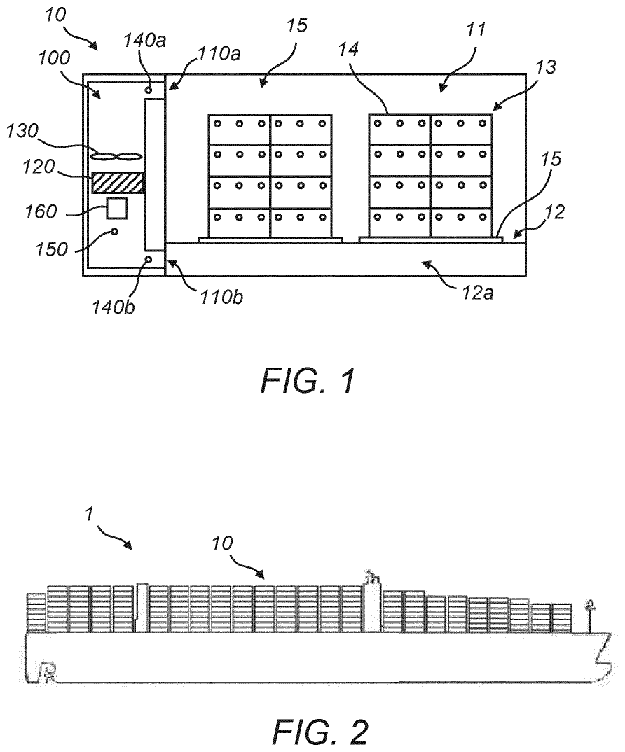

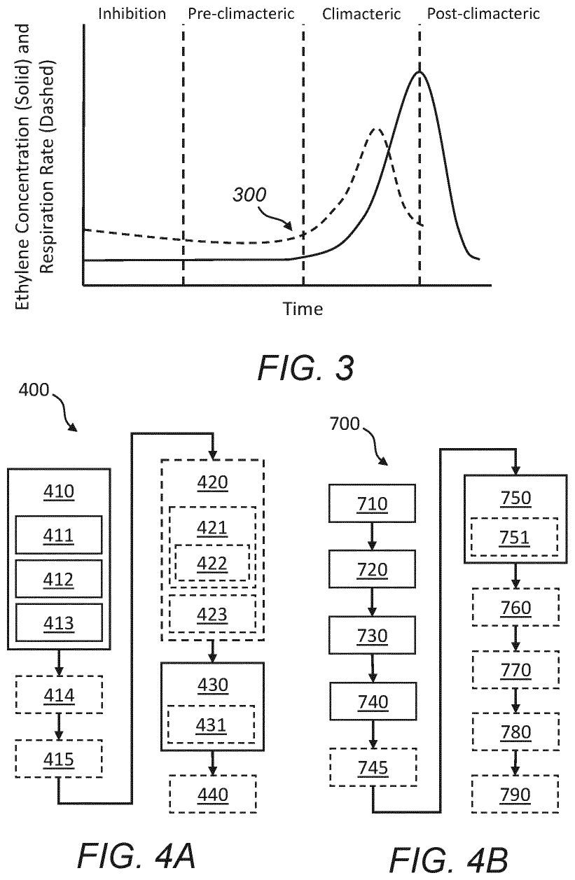

FIG. 1 shows an example storage unit, in the form of a transport unit, comprising a space 11, or “compartment” 11, for holding cargo 12, and an atmosphere control unit 100 for controlling an atmosphere in the compartment 11. For the purposes of the present example, the cargo 12 stored in the compartment 11 is ripenable produce, specifically “climacteric” produce, which is picked at a particular level of ripeness and comprises starch that can be converted to sugar during the ripening process. Examples of climacteric produce include bananas, avocados, plums, mangos, melons, apples, apricots, tomatoes, and other fruits or vegetables. The ripening process of climacteric produce is described in more detail below with reference to FIG. 3.

The cargo 12 is stacked and palletised. Specifically, the produce is stored in vented crates 14 which are stacked on top of one another and loaded onto pallets 15. The vented crates 14 allow gas in the compartment 11, such as provided by the atmosphere control system 100, to flow within and between the crates 14, and the climacteric produce stored therein. It also allows heat and gases released by the climacteric produce to escape into the compartment 11. This may reduce a likelihood of temperature hotspots arising in the cargo 12, which could lead to spoilage of the produce. The pallets 15 allow the cargo 12 to be readily loaded in to and out of the transport unit 10, such as by using plant machinery. It will be understood that in other examples the cargo 12 may be stored in the transport unit 10 in any other suitable way.

The transport unit 10 comprises a first port 110a and a second port 110b opening into the compartment 11. The first and second ports 110a, 110b open into the compartment 11 at spaced apart locations in the compartment 11. In other examples, the first and second ports 110a, 110b open into the compartment at any other suitable location in the compartment 11. In other examples, there is only one port 110a, or more than two ports 110a, 110b.

The atmosphere control system is coupled, or couplable, to the first and second ports 110a, 110b to pass gas, such as conditioned gas conditioned by the atmosphere control system 100, between the atmosphere control system 100 and the compartment 11. Specifically, in the present example, conditioned gas is supplied to the compartment 11 from the atmosphere control system 100 through either of the first and second ports 110a, 110b and return gas is returned to the atmosphere control system 100 through the other of the first and second ports 110a, 110b. In other examples, conditioned gas is supplied to the compartment 11 through either or both of the first and second ports 110a, 110b and either expelled into an external atmosphere external to the compartment 11 (i.e. the gas is not circulated through the compartment 11) or transport unit 10, and/or returned to the atmosphere control system 100 by any suitable mechanism, such as any suitable port, conduit or channel between the compartment 11 and the atmosphere control system 100. That is, gas is movable in the compartment 11 by operation of the atmosphere control system, without introducing gas from an environment external to the transport unit 10. In other examples, gas may be drawn from a location external to the transport unit 10, conditioned by the atmosphere control system 100, and supplied to the compartment 11 through either or both of the first and second ports 110a, 110b.

In the example shown, the transport unit 10 comprises a raised floor 12, the raised floor 12 defining a floorspace 12a between the raised floor 12 and a base of the transport unit 10. The first port 110a opens into a void 15 above the cargo 12 in the compartment 11, while the second port 110b opens into the floorspace 12a beneath the raised floor 12. In this way, conditioned gas, such as cooled gas, may be supplied into the void 15 above the cargo. The cooled gas may pass through the cargo 12 towards, and into, the floorspace 12a via vents in the raised floor 12. Alternatively, or in addition, conditioned gas may be supplied into the space 12a via the second port 110b to rise through the cargo 12 and into the void space 15, such as to be returned to the atmosphere control system 100 through the first port 110a. In other examples, no such raised floor 12 is present and/or the second port 110b opens into any other space in the compartment 11.

In some examples, the atmosphere control system 100 is configured to controllably switch between supplying gas to the compartment 11 through the first port 110a and supplying gas to the compartment 11 through the second port, such as by periodically switching, or by switching in response to sensed data, such as sensed temperatures of gas supplied and received by the atmosphere control system 100.

In the present example, the atmosphere control system 100 comprises a heat exchanger 120 for conditioning gas, specifically to adjust a temperature of gas flowing therethrough. The atmosphere control system 100 further comprises a fan 130 operable to draw gas past the heat exchanger 120. The fan 130 is also operable to move gas through the first and second ports 110a, 110b in any way as described above. Specifically, in the illustrated example, the fan 130 is operable in a first direction to cause return gas to pass from the compartment 11 through the second port 110b, across the heat exchanger 120 to condition the gas, and into the compartment 11 through the first port 110a. In other examples, the fan 130 is also operable in a second, reverse, direction to pass gas from the first port 110a to the second port 110b via the heat exchanger 120.

The atmosphere control system 100 of the illustrated example comprises first and second gas temperature sensors 140a, 140b. The first gas temperature sensor 140a is for sensing a temperature of gas flowing through the first port 110a. The second gas temperature sensor 140b is for sensing a temperature of gas flowing through the second port 110b. The first and second gas temperature sensors 140a, 140b are located in the atmosphere control system 100. In other examples, the first and second gas temperature sensors 140a, 140b are located in the respective first and second ports 110a, 110b, and/or in the compartment 11, such as in the void 15 above the cargo 12, in the floorspace 12a, or in closer proximity to the cargo 12. In other examples, there is more than one first gas temperature sensor 140a and/or more than one second gas temperature sensor 140b.

In some examples, temperatures sensed by the first and second gas temperature sensors are used to control the heat exchanger 120, such as by setting a target superheat of the heat exchanger 120, to supply conditioned gas at a particular temperature setpoint to the compartment 11. In other examples, the atmosphere control system 100 receives gas other than gas from the compartment 11, and the first and second gas temperature sensors 140a, 140b are configured to sense a temperature of gas received by the atmosphere control system 100.

In the illustrated example, the atmosphere control system 100 comprises a composition sensor 150 configured to sense a composition of gas passing through the respective first and second ports 110a, 110b. The composition sensor 150 is configured to detect a quantity of carbon dioxide (herein “CO2”) in the gas, such as a percentage of CO2 in the gas. In other examples, the composition sensor is configured to sense one or more other components of the gas, such as oxygen (herein “O2”), nitrogen, ethylene, or a competitive inhibitor for ethylene (an “ethylene blocker”), such as 1-methylcyclopropene (herein “1-MCP”). In some such examples, the composition sensor 150 is an array of composition sensors 150, each composition sensor 150 in the array being configured to sense the presence, and/or amount, of a single component of the gas.

The composition sensor 150 is shown in FIG. 1 as being located in the atmosphere control system 100 on the side of the heat exchanger 120 facing the second port 110b. In other examples, the composition sensor 150 may be located in any other suitable location in the atmosphere control system 100 or in the compartment 11, as with the temperature sensors 140a, 140b described above. In some examples, there is more than one composition sensor 150, each composition sensor 150 located in any suitable location in the atmosphere control system 100 or the compartment 11.

The atmosphere control system 100 of the present example further comprises a composition adjuster 160 configured to adjust the composition of gas flowing through, or by, the composition adjuster 160. The composition adjuster 160 of the present example comprises a CO2 scrubber configured to remove or reduce an amount of CO2 in the gas. In other examples, the composition adjuster 160 instead, or in addition, comprises any one or more of: an ethylene scrubber configured to remove or reduce an amount of ethylene in the gas; an ethylene injector configured to introduce or increase an amount of ethylene in the gas; and O2 scrubber configured to remove or reduce an amount of O2 in the gas; an O2 injector configured to introduce or increase an amount of O2 in the gas; and an ethylene-blocker injector, configured to introduce or increase an amount of an ethylene-blocker, such as 1-MCP, in the gas.

In some examples, the gas is selectively directed to flow through the composition adjuster 160, or a part thereof, such as by using any suitable arrangement of ducts, valves, and/or other flow control devices. In other examples, the composition adjuster 160 is merely located in the flow of gas, and/or is located in the atmosphere within the compartment 11. In the illustrated example, the composition adjuster 160 is a single unit located in the atmosphere control system 100. In other examples, the composition adjuster 160 may be located in any other suitable location in the atmosphere control system 100 or compartment 11, as with the temperature sensors 140a, 140b and the composition sensor 150 described above. In other examples, the composition adjuster 160 comprises plural injectors and/or scrubbers, each located in different locations in the atmosphere control system 100 and/or the compartment 11. In some examples, the composition adjuster 160, or a part thereof, is configured, or configurable, to pass gas from the atmosphere control system 100 and/or the compartment 11 to an exterior of the transport unit 10, such as into an ambient atmosphere. For example, the composition adjuster may be configured to pass CO2 and/or ethylene scrubbed from the gas into the ambient atmosphere. In other examples, the composition adjuster 160, or a part thereof, is configured, or configurable, to pass gas, such as ambient air, from the ambient atmosphere into the atmosphere control unit 100 and/or compartment 11.

In the present example, the atmosphere control system 100 is comprised in the transport unit 10. It will be appreciated that, in other examples, the atmosphere control system 100 is external to the transport unit. In some such examples, the transport unit comprises the first and second ports 110a, 110b to which the atmosphere control unit is coupled, or couplable, to supply gas to and/or to receive gas from the compartment 11. In other such examples, the atmosphere control system 100 is coupled, or couplable, to more than one such transport unit 10.

FIG. 2 shows an example marine vessel 1 comprising the transport unit 10. Specifically, the marine vessel 1 is a container vessel configured to transport plural such transport units 10. In some examples, the transport unit 10 is loaded on to the marine vessel 1 at a first port at a first time. The marine vessel then travels to a second port in a different location, arriving at the second port at a second time. The distance between the first and second ports may be such that a difference between the first and second times is greater than 2 days, such as up to 5 days, up to 8 days, up to 12 days, or more than 12 days. In some examples, the climacteric produce 12 in the transport unit 10 is loaded on to the marine vessel 1 at the first time in a particular ripened state, and arrives at the second port at the second time in either a different ripened state, or in substantially the same ripened state. That is, the atmosphere in the compartment 11 is controlled to control the ripening of the produce, such as to inhibit ripening of the produce, or to ensure the produce arrives at the second port in a particular ripened state.

An example ripening process of the produce will now be described in more detail in relation to FIG. 3. Climacteric produce is so named as it passes through a “climacteric” phase during ripening, in which a “respiration rate” of CO2 increases as starches stored in the produce are converted into sugars. The respiration rate (herein “RR”) is a rate of production of CO2 per kilogram of produce in the container, and can be calculated as follows:

$$RR = \frac{{DC}_{{CO}_{2}} \cdot V}{M} .$$

Here, DC_{CO}_{₂} is a time rate of change of the concentration of CO2 in the gas, such as sensed by the composition sensor 160, and represented as a percentage change per hour (%/hr) of CO2 in the gas; V is a volume of the gas in the transport unit 10, such as given by a total free volume of the compartment 11 and/or the atmosphere control system 100, such as represented in units of ml; and M is a mass of produce in the compartment 11, represented in units of kg. The respiration rate of CO2 therefore has units of ml kg⁻¹ h^{r-1}. In other examples, the RR, or any of the values used to determine the RR, may be provided in any other suitable units. For instance, DC_{CO}_{₂} could be a % change per second, or per day, while V could be represented in litres.

The mass M and volume V are determined in any suitable way. The mass M may, for instance, be determined by consulting a bill of laden of the transport unit 11, which may detail an amount and/or type of cargo 13 loaded in the compartment 10. Alternatively, or in addition the transport unit 11 may be weighed before transport, when fully laden, and the mass M may be deduced from a known unladen weight of the transport unit 11. In other examples, a weight sensor (not shown) is provided in the compartment 11 or in the floor 12. The volume V may similarly be inferred by consulting a bill of laden, or by otherwise determining an amount of free space in the compartment 11 after loading the cargo 13 into the compartment 11.

The ripening of climacteric produce is controlled by ethylene, which is a plant hormone highly synthesised during late stages of ripening. FIG. 3 shows, with a solid line, a concentration of ethylene in an atmosphere surrounding an example climacteric produce, and with a dashed line, a respiration rate of the climacteric produce such as avocados. The graph is split into three stages, or phases, which represent three different states of the climacteric produce. These states are: pre-climacteric, climacteric and post-climacteric.

In the pre-climacteric phase, a production of ethylene in the climacteric produce may be auto-inhibited. That is, in the pre-climacteric phase, ethylene in the produce may restrain, or inhibit, its own biosynthesis. This can prevent the produce from ripening immediately after it has been picked. As such, during the pre-climacteric phase, the ethylene concentration and respiration rate may remain relatively low.

The pre-climacteric phase can be maintained for long periods of time by controlling an atmosphere in the compartment, specifically by any one or more of: reducing a temperature in the compartment 11; reducing an amount of ethylene in the compartment 11; reducing an amount of O2 in the compartment 11; increasing an amount of CO2 in the compartment 11; and increasing an amount of ethylene-blocker in the compartment 11.

Towards the end of the pre-climacteric phase, physiological changes in the produce may cause ethylene production to become auto-catalytic. That is, at some point, a presence of ethylene may cause more ethylene to be produced. This can signal the start of the climacteric phase, in which ethylene production continues to increase, as shown in FIG. 3. This is accompanied by an increase in the respiration rate, as also shown in FIG. 3. The climacteric phase can be actively triggered to initiate ripening of the produce, such as by any one or more of: increasing a temperature in the compartment 11; increasing an amount of O2 in the compartment 11; reducing an amount of CO2 in the compartment 11 increasing amount of ethylene in the compartment; and reducing an amount of ethylene-blocker in the compartment 11.

In the example shown, the concentration of ethylene peaks at the end of the climacteric phase, signifying the start of the post-climacteric phase, and then starts to drop to at or below pre-climacteric levels. In the post-climacteric phase, the ripening process has ended, and fruit senescence has begun. That is, during the post-climacteric phase, the produce may begin to spoil.

It will be appreciated that the process shown in FIG. 3 is provided by way of example only, and that the actual process may vary depending on various factors, such as the type and/or quantity of produce, and/or conditions in the compartment 11. For example, the peaks of respiration rate and ethylene concentration in FIG. 3 may be of different heights, and/or may be shifted temporally. Alternatively, an increase in ethylene concentration may precede an increase in respiration rate and vice versa, and/or a peak in ethylene concentration may precede a peak in respiration rate and vice versa. Other such ripening processes will be known to a person skilled in the art.

In any event, it can be desirable to maintain the climacteric produce in an unripened, and/or a pre-climacteric state during transport, such as for an entire journey, or merely until ripening is to be initiated, such as to ensure the produce arrives at the second port in a particular ripened state. Premature ripening of the produce, such as due to the produce entering the climacteric stage too soon, may cause some or all of the produce to arrive in the second port in an overly-ripe, or even spoiled, state.

In the present example, as shown in FIG. 3, before the climacteric stage begins, there is a detectable (and often sudden) increase in the respiration rate, as shown by the arrow 300 in FIG. 3. This increase in respiration rate precedes the autocatalytic increase in ethylene production seen in the climacteric phase. In accordance with the present invention, actions can be performed to facilitate maintenance of, such as to maintain, or to attempt to maintain, the unripened state of the produce in response to such an increase being detected. Specifically, the actions are performed to in an attempt to maintain the produce in the pre-climacteric state, but may also be performed in an attempt to maintain the unripened state once the produce has entered the climacteric state. As described in more detail below, such actions may be to increase a flow rate of gas in the compartment 11, reduce a temperature in the compartment 11, reduce a concentration of O2 or ethylene in the compartment 11, or to increase a concentration of ethylene-blocker in the compartment.

FIG. 4A shows an example such method 400 for maintaining the climacteric produce in the unripened state, and specifically in the pre-climacteric state. The method comprises monitoring 410 the respiration rate of the climacteric produce, and in response to a change in the monitored respiration rate, specifically an increase in the monitored respiration rate in the present example, causing 430 an action to be performed to facilitate maintenance of the pre-climacteric state.

In some examples, the monitoring 410 the respiration rate comprises monitoring 411 an amount of CO2 in the gas in the compartment 11, or in the atmosphere control system 100, such as using the composition sensor 160. In some examples, the monitoring 411 the amount of CO2 in the gas comprises monitoring a rate of change in concentration of CO2 in the gas, which is used to determine the monitored respiration rate, such as using the equation above. In other examples, the respiration rate can be monitored directly using a sensor, such as the composition sensor 160.

In some examples, the method comprises determining 414 a leakage rate of gas into and/or out of the compartment 11, such as due to pressure differences between parts of the compartment 11, and/or the atmosphere control system 100, and an atmosphere external to the storage unit 10. Such leakage may occur through gaps in the storage unit, such as in or around a door of the storage unit, near apertures through which parts of the atmosphere control system 100, such as refrigerant components, pass between an internal space of the storage unit 10 and the external atmosphere, or near any other gaps in the storage unit 10. The leakage rate can be determined by monitoring a change in CO2 and a change in O2, such as sensed by the composition sensor 160, which as described above may comprise a CO2 sensor and an O2 sensor. Alternatively, the storage unit 10 may comprise separate CO2 and O2 sensors.

For instance, a rate of production of CO2 sensed by the composition sensor 160 may be representative of an actual rate of production of CO2 by respiration of the produce (i.e. the actual respiration rate, RR), minus a CO2 leakage rate, which is a rate of CO2 leaking out of the compartment 11. Meanwhile, a rate of decrease in O2 sensed by the composition sensor 160 may be representative of a rate of consumption of O2 by the produce, minus an O2 leakage rate, which is a rate of O2 leaking into the compartment from an external atmosphere. This can be represented by the following set of equations:

$${\dot{CO 2}}_{increase} = RR \cdot M - X \cdot CO 2 \% , \\ {\dot{O 2}}_{decrease} = O {2}_{consumption} - X \cdot O 2 \% ,$$

where M is the mass of produce in the storage unit, as above, X is the leakage rate, CO2_{increase} and O2_{decrease} are respective rates of increase/decrease of CO2% and O2% as sensed by the composition sensor(s) 160, CO2% is a concentration of CO2 in the compartment 11, as sensed by the sensor 160, and O2% is a concentration of O2 in an atmosphere external to the storage unit 10, which in many cases can be assumed to be around 20.8%. Typically, the respiration stoichiometry of produce is close to 1:1, meaning that the respiration rate (RR) and the O2 consumption of the produce can in many cases be assumed to be equal. Solving for X leads to the following equation for the leakage rate:

$$X = \frac{{\dot{O 2}}_{decrease} - {\dot{CO 2}}_{increase}}{( CO 2 \% - 20.8 \% )}$$

In some examples, the method comprises calibrating 415 the monitored respiration rate, such as by using the determined leakage rate X. For example, the calibrated monitored respiration rate may be calculated as the monitored respiration rate (CO2_{increase}) plus the CO2 leakage rate (which may herein be referred to as a “calibration factor”), which is a multiple of the leakage rate X and the CO2 concentration in the compartment 11, as follows:

$${RR}_{cal} = ( {\dot{CO 2}}_{increase} + X \cdot CO 2 \% ) / M .$$

It will be understood, however, that the above calculation is exemplary only, and the calibration factor and/or calibrated respiration rate may be determined in any other suitable way.

In the present example, the respiration rate is continuously monitored. In other examples, the monitoring 410 is intermittent, and/or is performed over a predetermined period of time. In the present example, the monitored respiration rate is an instantaneous respiration rate. A change in the respiration rate may therefore be an instantaneous change in the respiration rate. In other examples, the monitored respiration rate is a time-averaged respiration rate, such as averaged over a predetermined time period. The change in the respiration rate may therefore be a change in a time-averaged respiration rate, monitored over two or more time periods. In other examples, the monitored respiration rate is a monitored rate of change of the respiration rate of the climacteric produce. The change in the respiration rate may therefore be change in the rate of change of respiration rate. Such a change to, for example, a high rate of change of respiration rate may indicate a sudden change in respiration rate of the climacteric produce, such as a sudden increase, which as described above may precede the climacteric produce entering the climacteric phase.

In some examples, the monitored respiration rate is an accumulated respiration rate. That is, the monitoring 410 may comprise monitoring 410 the respiration rate over a time period and obtaining an area under a curve of the respiration rate vs. time in that time period. The accumulated respiration rate may therefore represent an accumulated amount of CO2 per kilogram of the climacteric produce that has been respired by the climacteric produce. An increase in the amount of CO2 produced may be indicative of an increase in the respiration rate, and possibly an upcoming transition of the produce into the climacteric state.

The monitored respiration rate is a current, or recent, respiration rate, such as to provide an up-to-date picture of the ripening stage of the produce. In other examples, a recent respiration rate may be a respiration rate monitored up to one minute, up to two minutes, or up to five minutes in the past. In other examples, the monitored respiration rate may be an older rate of change of respiration rate, particularly if the respiration rate is monitored intermittently, such as in intervals of greater than 5 minutes.

In some examples, the method comprises determining 420 the change in the respiration rate. In some examples, the determining 420 the change in the respiration rate comprises determining 423 whether the respiration rate has exceeded a threshold. In some such examples, the causing 430 the action to be performed is in response to the monitored respiration rate exceeding the threshold. The threshold may be a predetermined value of the monitored respiration rate or a threshold deviation from a predetermined baseline respiration rate, or from a time average value of the monitored respiration rate.

In some examples, the threshold deviation is a standard deviation, such as two or three standard deviations from the time-averaged respiration rate.

In some examples, the threshold is predetermined. In other examples, the method comprises determining 421 the threshold. In some such examples, the determining 421 the threshold comprises determining 422 a quantity and/or quality, such as a type, of the climacteric produce in the compartment 11. As suggested above, in some examples this can be by consulting a bill of laden, and/or using sensors, such as weight sensors, installed in the transport unit 10. In other examples, information on the quality and/or quantity of the climacteric produce is provided manually, such as by an operator, and the determining 422 the quality and/or quantity comprises consulting 422 the manually-input information. In some examples, the threshold is determined 421 based on the monitored respiration rate itself. For example, a higher average respiration rate of the produce may permit a higher permissible instantaneous respiration rate.

In some examples, the causing 430 the action to be performed comprises generating 431 a control signal based on the change in the monitored respiration rate, and causing 430 the action to be performed using the control signal.

In some examples, any one of a number of actions may be caused in response to the change in the respiration rate, such as: issuing a visual and/or audible alarm, or notification; increasing a flow rate of gas within the compartment 11, such as by causing an increase in a speed of the fan 130 of the atmosphere control system 100; reducing a temperature in the compartment 11, such as by causing a reduction in a temperature set point of the heat exchanger 120 of the atmosphere control system 100; reducing an amount of O2 or ethylene in the compartment 11, such as by causing operation of the composition adjuster 160; and increasing an amount of ethylene-blocker or CO2 in the compartment 11, such as by causing operation of the composition adjuster 160. As such, the causing 430 the action may comprise providing instructions to the transport unit 10, or the atmosphere control system 100, to cause the transport unit 10 or the atmosphere control system 100 to perform the action.

The change in respiration rate may be caused by temperature hotspots in the compartment 11, wherein produce in proximity to the hotspots may start ripening, or may have entered, or are at risk of entering, the climacteric state. Increasing a flow rate of gas within the compartment 11 and/or reducing a temperature in the compartment 11 can provide a more even distribution of temperature within the compartment. This can be done in an attempt to eliminate such hotspots in the compartment 11. This is to prevent further ripening of the produce in proximity to the hotspot, and/or to prevent more widespread ripening of the produce in the compartment, even if it was not possible to prevent localised ripening. Similarly, changing a composition of the gas in the compartment 11, such as by reducing an amount of O2 and/or ethylene in the compartment 11, or increasing an amount of ethylene blocker or CO2 in the compartment 11, can also inhibit ripening, or further ripening, of the produce in the compartment 11. Causing 430 such actions may therefore facilitate maintenance of the produce in the unripened state, and preferably in the pre-climactic state for climacteric produce.

In some examples, issuing the alarm, or notification, may comprise notifying an operator, or maintenance personnel. This can allow action to be taken by the operator or maintenance personnel to maintain the produce in the unriprened state, particularly if the increase in respiration rate is due to a failure of the transport unit 10 and/or atmosphere control system 100 to maintain a suitable atmosphere in the compartment 11 to inhibit ripening. In this way, the operator or maintenance personnel can resolve any issues, and/or arrange for any issues to be resolved, in an attempt to maintain the unripened state of the produce. Alternatively, or in addition, the issuing the alarm or notification may be to notify a recipient of the produce that the produce is at risk of ripening, or that action needs to be or has been taken to facilitate maintenance of the unripened state of the produce. This may allow the recipient to consider sourcing alternative produce in case the produce in the transport unit spoils and/or is delivered in an undesired state of ripeness, such as due to the produce entering the climacteric state despite performance of the method 400.

In some examples, the method 400 further comprises causing 440 one or more of: modification of a delivery parameter; forecasting a cargo claim. Modification of the deliver parameter may comprise adjusting a time for delivery of the transport unit 10 to a recipient, such as to a recipient at the second port discussed above. In some examples, this is by indicating, such as by providing a signal to any suitable system or operator, that the transport unit should be unloaded from the vessel 1, and/or delivered to its recipient, or redirected to a new recipient, earlier than it might otherwise have been. In some examples, the causing 430 the action to issue an alarm comprises issuing the signal to modify the transport parameter. This may aid with prolonging a shelf-life of the produce, such as by prioritising shipment of that container, which may increase a likelihood that the produce is shipped in an unripened state, or, if it was not possible to maintain the unripened state, that it is shipped at a reduced level of ripening. Redirecting the container to a different recipient may reduce wastage of the produce, such as might otherwise occur if the produce was delivered to the intended recipient at an undesirable level of ripeness. For instance, the produce might instead be redirected to a recipient at an earlier port stop of the marine vessel, or to a recipient having a shorter onward chain for the produce. Forecasting a cargo claim may comprise determining, such as using a statistical analysis or otherwise, the likelihood of a recipient of the produce raising a claim, such as due to spoilage of the produce and/or delivery in an undesired state of ripeness, as described above.

In some examples, the calibrating 415 the monitored respiration rate comprises determining a calibrated respiration rate using the example method shown in FIG. 4B. Specifically, FIG. 4B shows an example method 700 for determining a respiration rate, such as by using only a CO2 sensor, and not also an O2 sensor, as discussed above. The method 700 comprises moving 710 gas in the compartment 11 at a first flow rate, such as by operating the fan 130 at a first speed, and measuring 720 a first respiration rate of the produce in the compartment 11. The method further comprises moving 730 gas in the compartment 11 at a second flow rate, greater than the first flow rate, such as by operating the fan 130 at a second speed, greater than the first speed, and measuring 740 a second respiration rate of the produce. The method further comprises determining 750 a calibration factor of based on the measured first and second respiration rates. In some examples, the method further comprises determining 770 a calibrated respiration rate based on the calibration factor.

In some examples, the respiration rate calibrated in FIG. 4B is the monitored respiration rate described above with reference to FIG. 4A. That is, in some examples, the calibrating 415 the monitored respiration rate, as in FIG. 4A, comprises determining 760 a calibrated monitored respiration rate based on a calibration factor determined as in FIG. 4B. In other examples, the entire ripening process of the produce may be controlled using a calibrated respiration rate determined as in the method 700 of FIG. 4B. That is, a rate of ripening of produce in the pre-climacteric, climacteric and/or post-climacteric state may be controlled using such a calibrated respiration rate.

Determining 760 a calibrated respiration rate in accordance with the method 760 of FIG. 4B may be particularly advantageous in storage units 10 which do not comprise curtains, or other components, for better sealing the compartment 11 from the external atmosphere. Many such storage units 10 may not normally comprise both CO2 and O2 sensors 160. The method 700 shown in FIG. 4B may therefore be used to determine a more accurate respiration rate in especially “leaky” storage units 10, without the expense of installing additional O2 sensors. It will be understood, however, that either of the methods 400, 700 shown in FIGS. 4A and 4B could be applied to any suitable storage unit 10, regardless of a level of sealing of the compartment 11 from the external atmosphere.

In the illustrated example, the first flow rate is zero, or close to zero, so that a pressure difference between the compartment 11 and an exterior of the storage unit 10 is zero, or close to zero. This may limit a leakage of gas into or out of the compartment 112 through any gaps in the storage unit 10 due to such a pressure difference. Movement of the gas at the second, higher, flow rate, may result in pressure differences between at least a part of the compartment 11, and/or the atmosphere control system 100, and the exterior of the storage unit 10, causing a leakage of gas into and/or out of the compartment 11 and/or the atmosphere control system 100. For example, a reduced pressure may arise at a return side of the fan 130, such as in proximity to the second port 110b, while in increased pressure may arise downstream of the fan 130, such as in proximity to the first port 110a, or in the compartment 11, such as near a door of the storage unit 10. In some examples, a fan is provided in the compartment 11 to move the gas in the compartment 11, and a pressure in the compartment 11 may be changed by operation of the fan at the second speed. In some examples, the first flow rate is achieved by uncoupling the atmosphere control system 100 and the compartment 11, such as by closing either or both of the first and second ports 110a, 110b. This may be advantageous when, for instance, the fan 130 is operated when defrosting the heat exchanger 120, such as to blow melted ice from the heat exchanger 120. In other examples, the atmosphere control system 100 is external to the storage unit 10, and may be physically uncoupled from the storage unit 10, such as when the storage unit 10 is transported and/or loaded onto a marine vessel 1.

In some examples, the method comprises determining 765 a speed correlation factor, or flow rate correlation factor, and the calibration factor and/or the calibrated respiration rate is determined based on the speed correlation factor. The speed correlation factor may account for changing pressure differences and/or leakage rates as the flow rate of gas in the compartment 11, and/or the speed of the fan 130, is varied. For instance, where the respiration rate is monitored when gas is moving in the compartment 11 at a third flow rate, which is higher than the second flow rate, then a calibration factor determined based on the first and second respiration rates may be not be as accurate when used to calibrate the monitored respiration rate as, for example, a calibration factor determined based on the first respiration rate and the monitored respiration rate might be. In such a case, the speed correlation factor may be applied to calibrate the monitored respiration rate more accurately, for example to account for a greater pressure difference and higher leakage rate when gas is moved in the compartment 11 at the third flow rate, and/or the fan 130 is operated at a third speed, compared to when gas is moved in the compartment 11 at the second flow rate and/or the fan 130 is operated at the second speed.

It will be appreciated that the moving 710 the gas at the first flow rate and measuring 720 the first respiration rate may be performed before, or after, the moving 730 the gas at the second flow rate and measuring 740 the second respiration rate. As such, generally speaking, the calibration factor is determined by changing a flow rate of gas in the space from a relatively high flow rate to a relatively low flow rate, or vice versa, and determining a calibration factor based on respiration rates measured at the respective high and low flow rates. The high and low flow rates may be “high” and “low” relative to each other, so that at the high flow rate, a pressure difference may be present between at least a part of the compartment 11 and an external atmosphere, and at the low flow rate, such a pressure difference is minimal, or negligible.

In this way, the leakage rate X of gas into and/or out of the compartment 11 may be represented as a ratio of the measured respiration rate at the high flow rate and the measured respiration at the low flow rate. In some examples, the calibration factor is determined as the leakage rate X multiplied by the CO2 concentration in the compartment 11, such as measured by the composition sensor 160. This may be added on to the respiration rate sensed by the composition sensor 160 to determine the calibrated respiration rate, in a similar way as discussed above. Calibrating 770 the respiration rate in this way can avoid the need for both a CO2 and an O2 sensor in the storage unit 10, reducing up-front cost and/or maintenance costs of the storage unit 10. In some examples, the leakage rate X is between 0.1 m3 h^{r-1} and 1 m3 h^{r-1}, but alternatively the leakage rate X may be any other value.

The determining 750 the calibration factor may be performed multiple times. For example, the determining 750 the calibration factor may comprise updating 751 the calibration factor, such as by replacing the calibration factor with a new calibration factor, or by adjusting the calibration factor based on a new calibration factor.

In some examples, the method 700 comprises determining 750, and/or updating 751, the calibration factor when one or more predetermined conditions have been met, such as: the storage unit being loaded onto a container ship; the atmosphere control system or a part thereof being inoperable, or uncoupled from the space; an atmosphere in the space reaching a predetermined temperature; an atmosphere in the space reaching a predetermined composition; an atmosphere in the space being stable; the passage of a predetermined period of time since the calibration factor was last determined; and a change in an external temperature and/or pressure reaching a predetermined threshold. In some examples, the method comprises determining 745 whether the one or more predetermined conditions have been met.

For instance, the fan 130 may not be operated, or may be precluded from operating, when loading the storage unit 10 onto the marine vessel 3, providing an opportunity to determine or update the calibration factor, In some examples, a “stable atmosphere” means that a temperature, composition, and/or any other suitable parameter of the atmosphere in the compartment 11 has remained relatively unchanged for a predetermined period of time. This may allow a more accurate calibration factor to be determined. Ensuring that a predetermined temperature and/or composition has been reached in the compartment 11 may ensure that the cargo 13 is, for example, sufficiently cooled before turning the fan 130 off. The cargo 13 may, for example, be cooled below a set temperature for the cargo 13 to allow the compartment 11 to be uncooled for a period of time without causing spoilage, or premature ripening, of the produce. Alternatively, or in addition, a level of ethylene and/or O2 in the compartment may be reduced to facilitate maintenance of an unripened state of the produce while the fan 130 is inoperable.

In other examples, the calibration factor can be determined and/or updated periodically, such as whenever the fan 130 is turned off or on, or at repeat intervals of time, such as up to 15 minutes, up to 30 minutes, up to 1 hour, up to 2 hours, up to 12 hours, up to 1 day, or more than 1 day, optionally subject to other predetermined conditions described above being met. This may allow the calibration factor to be regularly updated to provide a more accurate calibrated respiration rate. In other examples, the calibration factor can be determined whenever there is a significant change in an external, or ambient, temperature and/or pressure. This may account for increased or reduced leakage rates caused by a change in pressure difference between the compartment 11, or a part thereof, and the external atmosphere of the storage unit 10.

In some examples, the method 700 comprises storing 780 the calibration factor and/or the calibrated monitored respiration rate, such as in a computer-readable memory. Alternatively, or in addition, the method 700 comprises transmitting 790 a signal indicative of the calibration factor and/or the calibrated monitored respiration rate, to the storage unit 10 and/or to the atmosphere control system 100.

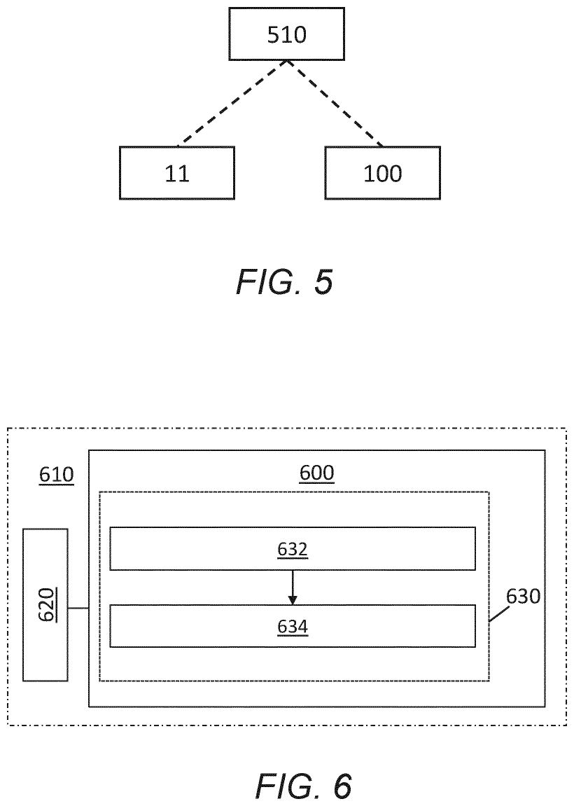

Turning now to FIG. 5, shown is a control system 500 comprising a controller 510, the transport unit 10 and the atmosphere control system 100. The controller 510 is configured to perform the method 400 described above. In the present example, the controller 510 is a remote controller, such as comprised in the marine vessel 1, or in a cloud-based computing system, and is communicatively coupled, or couplable, to the transport unit 10 and the atmosphere control system 100, such as to respective controllers thereof. In this way, the controller 510 is configured to cause 430 the transport unit 10 and/or the atmosphere control system 100 to perform one or more of the actions of the method 400 described above. In other examples, the control system 500 comprises only the controller 510 coupled to the transport unit 10, or only the controller 510 coupled to the atmosphere control system 100. In other examples, the controller is comprised in the transport unit 10 and/or the atmosphere control system 100. In some such examples, the controller is configured to control the transport unit and/or the atmosphere control system. In some examples, the controller 510 is configured to perform one or more of the actions of the method 400 itself. In other examples, the transport unit 11 and the atmosphere control system 100, or respective controllers thereof, are communicatively coupled, or couplable, to each other.

FIG. 6 shows a schematic diagram of a non-transitory computer-readable storage medium 600 according to an example. The non-transitory computer-readable storage medium 600 stores instructions 630 that, if executed by a processor 620 of a controller 610, cause the processor 620 to perform a method according to an example. In some examples, the controller 610 is the controller 510 as described above with reference to FIG. 5 or any variation thereof discussed herein. The instructions 630 comprise: monitoring 632 a respiration rate of produce in a storage unit 11; and in response to a change in the monitored respiration rate, causing 634 an action to be performed to facilitate maintenance of an unripened state of the produce. In other examples, the instructions 630 comprise instructions to perform any other example method described herein, such as the method 400 described above with reference to FIG. 4.

It will be appreciated that any two or more of the above described examples may be combined, and/or that any of the features of one example may be combined with any of the features of one or more other examples, in any suitable way. Additionally, examples of the present invention have been discussed with particular reference to the examples illustrated. It will be appreciated that variations and modifications may be made to the examples described within the scope of the invention as defined by the appended claims.

1. A method for maintaining produce in an unripened state in a storage unit, the storage unit comprising a space in which the produce is stored, the method comprising:
monitoring a respiration rate of the produce; and
in response to a change in the monitored respiration rate, causing an action to be performed to facilitate maintenance of the unripened state.

2. The method of claim 1, wherein the monitored respiration rate comprises at least one of:
an instantaneous respiration rate of the produce;
a rate of change of a respiration rate of the produce;
a time averaged respiration rate of the produce; or
an accumulated respiration rate of the produce over a predetermined period of time.

3. The method of either claim 1, wherein the causing the action to be performed is in response to the monitored respiration rate exceeding a threshold.

4. The method of claim 1, wherein the action comprises at least one of:
issuing an alarm;
increasing a flow rate of gas within the space;
reducing a temperature in the space;
reducing an amount of oxygen in the space;
increasing an amount of carbon dioxide in the space;
reducing an amount of ethylene in the space; or
increasing an amount of ethylene blocker in the space.

5. The method of claim 1, further comprising, in response to the change in the respiration rate, causing at least one of:
modification of a delivery parameter; or
forecasting of a cargo claim.

6. A controller configured to perform the method of claim 1.

7. An atmosphere control system operable by the controller of claim 6, the atmosphere control system configured to control an atmosphere in the space of the storage unit, and to perform the action to facilitate maintenance of the unripened state of the produce.

8. The atmosphere control system of claim 7, comprising a fan for controlling an amount of gas supplied to the space, wherein the increasing a flow of gas within the space comprises increasing a speed of the fan.

9. The atmosphere control system of claim 7, comprising a heat exchanger configured to adjust a temperature of gas supplied to the space, wherein the reducing the temperature in the space comprises reducing a temperature setpoint of the heat exchanger.

10. A storage unit comprising, or couplable to, the atmosphere control system of claim 7, the storage unit comprising the space in which the produce is stored.

11. A marine vessel comprising the atmosphere control system of claim 7.

12. A method of determining a respiration rate of produce in a storage unit, the storage unit comprising a space in which the produce is stored, the space being couplable to an atmosphere control system that is operable to move gas in the space, the method comprising:
moving gas in the space at a first flow rate and measuring a first respiration rate of the produce;
moving gas in the space at a second flow rate, greater than the first flow rate, and measuring a second respiration rate of the produce;
determining a calibration factor based on the measured first and second respiration rates; and
determining a calibrated respiration rate based on the calibration factor.

13. The method of claim 12, wherein the first and second respiration rates are measured at respective first and second times, and wherein the method further comprises:
at a third time, after the first and second times, moving gas in the space at a third flow rate and measuring a third respiration rate; and
updating the calibration factor based on the third respiration rate.

14. The method of claim 12, comprising:
determining that one or more predetermined conditions have been met; and
in response to said determining that the one or more predetermined conditions have been met, determining the calibration factor,
wherein the one or more predetermined conditions comprise at least one of:
the storage unit being loaded onto a container ship;
the atmosphere control system or a part thereof being inoperable, or uncoupled from the space;
an atmosphere in the space reaching a predetermined temperature;
an atmosphere in the space reaching a predetermined composition;
an atmosphere in the space being stable;
the passage of a predetermined period of time since the calibration factor was last determined; or
a change in an external temperature and/or pressure reaching a predetermined threshold.

15. A method of controlling a ripening process of ripenable produce, the method comprising:
determining a calibrated respiration rate by carrying out the method of claim 12; and
controlling the ripening process based on the calibrated respiration rate.

16. A marine vessel comprising the atmosphere control system of claim 7.
